(19) United States
(12) Patent Application Publication
(10) Pub. No.: US 2016/0296741 A1
(43) Pub. Date: Oct. 13, 2016

(54) FLUID FLOW ASSEMBLY WITH RECEPTACLE CONNECTOR CLIP

(71) Applicant: Larry Patterson, Mackinaw, IL (US)

(72) Inventor: Larry Patterson, Mackinaw, IL (US)

(21) Appl. No.: 15/095,625

(22) Filed: Apr. 11, 2016

Related U.S. Application Data
(60) Provisional application No. 62/146,271, filed on Apr. 11, 2015.

(51) Int. Cl. A61M 39/10; A61M 25/00

(52) U.S. Cl. CPC A61M 39/1011; A61M 25/0017; A61M 2039/085

(57) ABSTRACT

A clip for retaining the connection of a socket fitting to a plug fitting of a receptacle connector. The clip includes a first connection end, a second connection end, and an elongated retaining portion. The first connection end include a tube aperture configured to be rotatably connected to one of a first tube and a second tube through having the tube inserted through the tube aperture. The second connection end includes a tube connector configured to be selectively connected around the other of the first tube and the second tube. The elongated retaining portion includes a first retaining end and a second retaining end, the first retaining end fixedly connected to and configured to bend in relation to the first connection end, and the second retaining end fixedly connected to and configured to bend in relation to the second connection end.

### PRIORITY

This application claims priority to and incorporates by reference in its' entirety, U.S. Provisional Patent Application No. 62/146271, entitled “Feeding Tube Clip System”, and filed Apr. 11, 2015.

### TECHNICAL FIELD

The present invention generally relates to fluid tube connectors for fluid tubes inserted into a living body and more particularly to clips to maintain the connection between two fluid tube connectors.

### BACKGROUND OF THE INVENTION

A feeding tube assembly may include a gastronomy tube and a nutrition tube detachably connected. When a patient is unable to eat solid food or swallow liquids, an implant end of the gastronomy tube may be surgically implanted in their stomach. The other, connecting end of the gastronomy tube may connect to the nutrition tube, through a receptacle connector. A nutrient source end of the nutrition tube may be connected to a source of nutrient, such as a bag. The other, connecting end of the nutrient tube may connect to the gastronomy tube. Nutrients, water, and/or medicines may be provided through the nutrition tube, to the gastronomy tube, and thus to the patient.

Other type tubes may also be inserted into a human body, such as catheters. Catheters may be inserted into a urethra and connected to a urine tube first end to remove urine from the urethra so the patient will not have to move. The urine tube may have a connection end which may be connected through a receptacle connector to a disposal tube connecting to a bag to collect and dispose of the urine.

The receptacle connector for either the gastronomy or urine tube may include a socket fitting connected to the gastronomy or urine tube on the connection end, and a plug fitting connected to the nutrition or disposal tube on the connection end. Sometimes the connection fittings may be reversed, or may take the form of similar connectors. The gastronomy tube and the nutrition tube may be disconnected and reconnected many times during a day to allow for patient movement, and regular replacement of the nutrition tube. The urine tube and disposal tube may likewise be disconnected numerous times. The fitting material may become fatigued. The connection made by the receptacle connector may be weakened by this fatigue.

When the connection at the receptacle connector is weakened, the fittings may loosen. Leaks of nutritional or other substances being fed to the patient through the feeding tube assembly may occur when the connection is weak. The connection itself may also be broken, especially when the patient moves or stomach gas flows into the gastronomy tube. Leaks of urine may occur in a catheter urine tube assembly may likewise occur. Nutritional material, urine, or other substances may flow onto the patient and bedding causing discomfort to the patient. Nursing and other medical personnel must spend precious time cleaning the patient and the bedding. Nutritional material may be wasted.

U.S. Patent Application Publication 2013/0338642 A1 filed by Maulin discloses a feeding tube clip with a first tube clasp. The tube clasp has a truncated cone shape with an opening along a length of the truncated cone shape. A frame has a first end and a second end. A clip end connector is attached to the second end of the frame. The clip end connector forms a groove perpendicular to a length of the frame. A clip end has a second tube clasp perpendicular to a mating slot. The mating slot mates to the clip end connector. The mating slot has an opening that aligns with the hole of the clip end connector. In addition a stop clip may be used to engage the clip end. The stop clip has a peg that inserts into the hole of the clip end connector and the aligned opening of the clip end. The stop clip ensures a very strong secure connection.

Clips made to keep receptacle connectors connected may have problems fitting around the many different types of receptacle connectors and tubes used by the medical industry if they are rigid. They may not be flexible enough to fit the size of the connector and flex around different features of the connector. The clips may not have the size and shape to accommodate the many styles of connectors and may be expensive to manufacture.

As can be seen, there may be an ongoing need for a clip to maintain the connection of receptacle connectors in use with tubes inserted into a living body which is flexible enough to be used with many styles and size of connectors. The disclosed clip, systems, and methods may solve the above or other problems.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

In one aspect of the present invention, a clip for retaining the connection of a socket fitting to a plug fitting of a receptacle connector, one of the socket fitting and the plug fitting fluidly connected to an insertion in a living body through an insertion tube at a first connection port, and the other of the socket fitting and the plug fitting fluidly connected to a fluid receptacle through a receptacle tube at a second connection port is disclosed. The clip includes a first connection end, a second connection end, and an elongated retaining portion. The first connection end includes a first tube aperture configured to be rotatably connected to one of the insertion tube and the receptacle tube through having the one of the insertion tube and the receptacle tube inserted through the first tube aperture. The second connection end includes a tube connector configured to be selectively and detachably connected around the other one of the insertion tube and the receptacle tube. The elongated retaining portion includes a first retaining end and a second retaining end, the first retaining end fixedly connected to and configured to bend in relation to the first connection end, and the second retaining end fixedly connected to and configured to bend in relation to the second connection end.

In another aspect of the present invention, a socket fitting for a receptacle connector is disclosed. The receptacle connector is for fluidly connecting an insertion in a living body with a fluid receptacle and includes a plug fitting, the plug fitting including a plug port and configured to be inserted into the socket fitting, the plug port configured to fluidly connect to the fluid receptacle through a receptacle tube. The socket fitting includes a first socket port, an outer surface, and a clip. The first socket port is configured to fluidly connect to the insertion through an insertion tube. the clip includes a socket connection end, a receptacle tube connection end, and an elongated retaining portion connecting the socket connection end to the receptacle tube connection end. The socket connection end is fixedly attached to the outside surface. The tube connection end is configured to be selectively and detachably connected to the receptacle tube.

In yet another aspect of the present invention, a fluid flow assembly for connecting an insertion within a living body with a fluid receptacle for collecting fluids through the insertion from the body or providing fluids through the insertion to the body is disclosed. The fluid flow assembly includes an insertion tube, a receptacle connector, a receptacle tube, and a clip. The insertion tube includes a first insertion tube end and a second insertion tube end. The first insertion tube end is fluidly connected with the insertion. The receptacle connector includes a body end, a receptacle end, a socket fitting, and a plug fitting. The socket fitting includes a socket and a socket port located on one of the body end and the receptacle end. The plug fitting includes a plug port and a plug inserted into and fluidly connected with the socket. The plug port is located at the other of the body end and the receptacle end. The receptacle tube includes a first receptacle tube end and a second receptacle tube end. The first receptacle tube end is fluidly connected with one of the socket port and the plug port. The one of the socket port and the plug port is located on the receptacle end. The second receptacle tube end is fluidly connected with the fluid receptacle. The clip includes a first connection end, a second connection end, and an elongated retaining portion. The first connection end includes a tube aperture rotably connected around one of the insertion tube at the second insertion tube end and the receptacle tube at the first receptacle tube end. The second connection end includes a tube connector configured to be selectively and detachably connected around the other of the insertion tube at the second insertion tube end and the receptacle tube at the first receptacle tube end. The elongated retaining portion includes a first retaining end and a second retaining end. The first retaining end is fixedly connected to and configured to bend in relation to the first connection end. The second retaining end is fixedly connected to and configured to bend in relation to the second connection end. The insertion tube second end is fluidly connected to the other of the socket port and the plug port. The other of the socket port and the plug port is located on the body end.

These and other features, aspects and advantages of the present invention will become better understood with reference to the following drawings, description and claims.

### BRIEF DESCRIPTION OF THE DRAWINGS

FIG. 1 is a perspective view of a fluid flow assembly from prior art.

FIG. 2 is a perspective view of a socket fitting of a receptacle connector, including a clip, fluidly connected to an insertion in a living body, according to an exemplary embodiment of the invention.

FIG. 3 is a perspective view of a first embodiment of a fluid flow assembly including the socket fitting and clip of FIG. 2, according to an exemplary embodiment of the invention.

FIG. 4 is a second perspective view of the fluid flow assembly of FIG. 3.

FIG. 5 is a perspective view of a second embodiment of a fluid flow assembly, according to an exemplary embodiment of the invention.

FIG. 6 is a second perspective view of the fluid flow assembly of FIG. 5.

FIG. 7 is a perspective view of a first embodiment of a clip, according to an exemplary embodiment of the invention.

FIG. 8 is a perspective view of a second embodiment of a clip, according to an exemplary embodiment of the invention.

FIG. 9 is a perspective view of a third embodiment of a clip, according to an exemplary embodiment of the invention.

FIG. 10 is a perspective view of a fourth embodiment of a clip, according to an exemplary embodiment of the invention.

FIG. 11 is a perspective view of a socket fitting of a receptacle connector from prior art.

FIG. 12 is a perspective view of a first embodiment of a socket fitting of a receptacle connector with a clip, according to an exemplary embodiment of the invention.

FIG. 13 is a perspective view of a second embodiment of a socket fitting of a receptacle connector with a clip, according to an exemplary embodiment of the invention.

FIG. 14 is a perspective view of a third embodiment of a socket fitting of a receptacle connector with a clip, according to an exemplary embodiment of the invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features. However, any single inventive feature may not address any of the problems discussed above or may only address one of the problems discussed above. Further, one or more of the problems discussed above may not be fully addressed by any of the features described below.

While the fluid flow assembly, socket fitting, and clip are described with reference to several illustrative embodiments described herein, it should be clear that the present invention should not be limited to such embodiments. Therefore, the description of the embodiments provided herein is illustrative of the present invention and should not limit the scope of the invention. In addition, while the following description references drawings showing particular configurations and proportions, it will be appreciated that the invention may be configured to have other configurations and proportions.

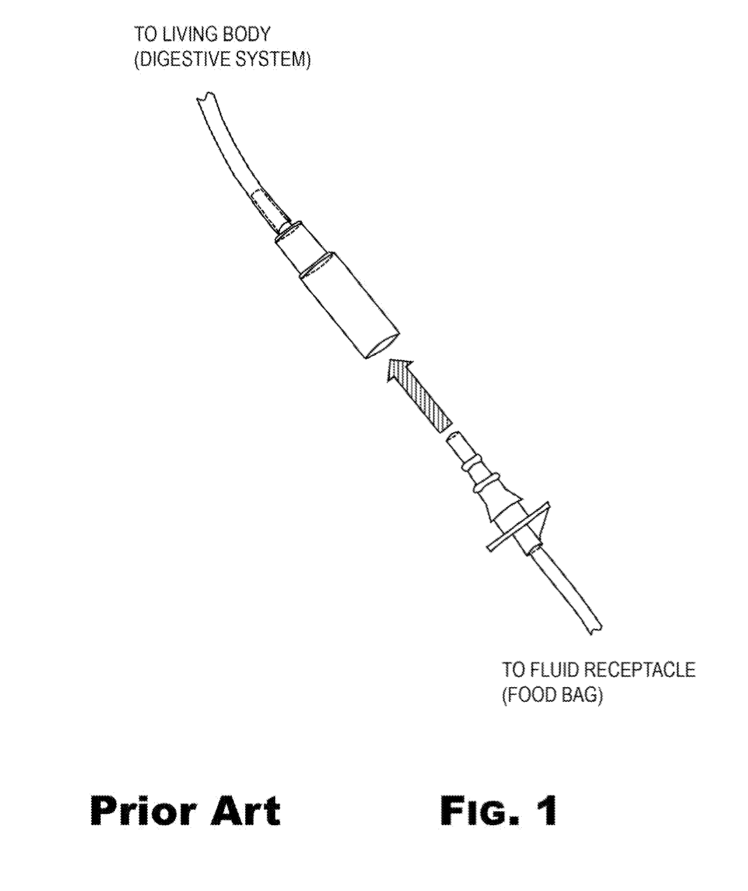

Referring now to FIG. 1, a fluid flow assembly, from the prior art, is illustrated in perspective view. It may be necessary to transfer fluids to, or transfer fluid from, a patient at times. For example, a patient may not be able to swallow food and may need to have an input end of a gastronomy tube surgically implanted in their stomach. Liquid nutrition may then need to be transferred through the input from a fluid receptacle, such as a bag. In another example, a patient may have a catheter inserted in the urethra when they are unable to move from a bed. Urine may need to be transferred through the catheter to a fluid receptacle, such as a bag, for disposal.

Many times the insertion (the gastronomy tube or catheter) may be fluidly attached to a tube and/or first part of a receptacle connector. The fluid receptacle may be fluidly connected to a tube and a second part of the receptacle connector. The first part of the receptacle connector may be a socket fitting, and the second part of the receptacle may be a plug fitting. The plug fitting may be inserted into the socket fitting to fluidly connect the fluid receptacle to the insertion. The plug fitting may be detached from the socket fitting to change the fluid receptacle. For example, when the liquid nutrition has all transferred to the patient, or for a catheter, when the fluid receptacle is full and must be disposed of.

Although illustrated with the socket fitting fluidly connected to the insertion and the plug fitting fluidly connected to the fluid receptacle, this may be reversed. Over time, as the receptacle connector is used, it may wear. This may result in the socket fitting and plug fitting accidentally detached, and fluid flowing onto the patient and their bedding. In addition, movement from the patient may accidentally detach the socket fitting from the plug fitting. The fittings could be fixedly attached, but that could cause harm to patient if they moved such that the insertion was pulled out of their body. There is a need for a fluid flow assembly where the receptacle connector will not accidentally disconnect, but where if the patient moves too far, will disconnect to avoid an insertion being pulled from their body.

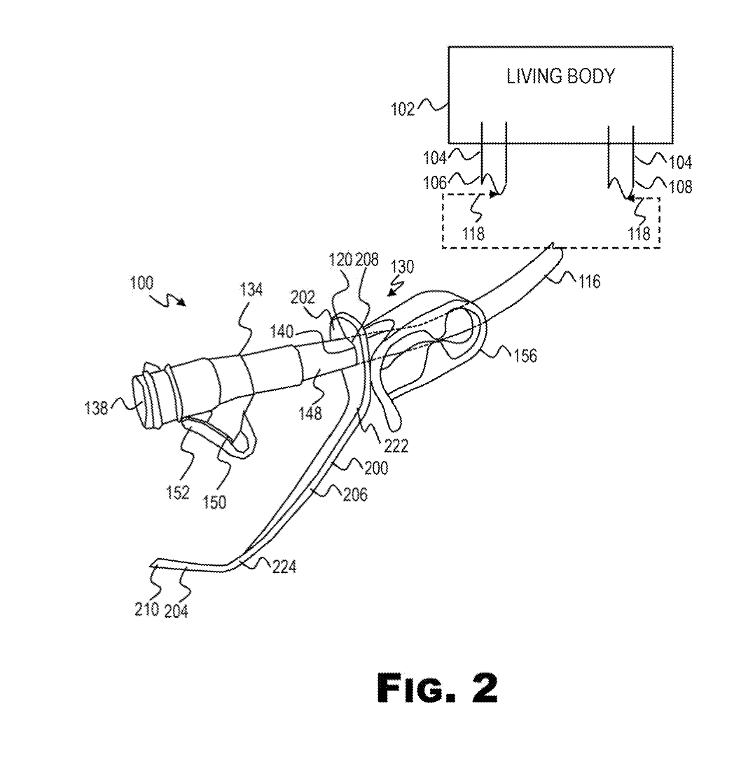

Referring now to FIG. 2, an exemplary fluid flow assembly 100 is partially illustrated in a perspective view. FIG. 2 illustrates of a socket fitting 134 of a receptacle connector 128, including a clip 200, fluidly connected to an insertion 104 in a living body 102, according to an exemplary embodiment of the invention. The living body 102 may be a human patient, but is not limited to a human. Non-limiting examples of the insertion 104 include an input end 106 of a gastronomy tube, and a catheter 108. The insertion 104 may be fluidly connected to an insertion tube 116. The insertion tube 116 may include a first insertion tube end 118 fluidly connected to the insertion 104 and a second insertion tube end 120 fluidly connected to the receptacle connector 128. The insertion tube 116 may be a long, hollow cylinder of plastic or another flexible material or any other connector known in the art for transferring fluids to and from the living body 102.

The receptacle connector 128 may be any connector known in the art for connecting an insertion tube 116 from a living body 102 to a fluid receptacle 110 (shown and described in relation to FIG. 3). The fluid receptacle 110 may include a body end 130 and a receptacle end 132 (shown and described in relation to FIG. 3). The second insertion tube end 120 may be connected to the receptacle connector 128 at the body end 130. The receptacle connector 128 may include a socket fitting 134 and a plug fitting 136 (shown and described in relation to FIG. 3). In the illustrated embodiment, the body end 130 is located on the socket fitting 134. However, in other embodiments, the body end 130 may be located on the plug fitting 136,

The socket fitting 134 may include a socket 138, a first socket port 140, and an outer surface 148. The socket 138 may include a hollow into which the plug fitting 136 may be inserted. The first socket port 140 may include an aperture through which fluid flowing from or to the insertion tube may pass. The socket fitting 134 may include a channel (not shown for flow fluids from the first socket port 140 to the socket 138 for transfer to the plug fitting 136.

The socket fitting 134 may also include a second socket port 150. When the fluid flow assembly 100 is being used to provide fluids through an insertion 104, the second socket port 150 may be connected to another receptacle for providing other fluids to the living body 102. For example, medication may be provided through the second socket port 150. The socket fitting 134 may include a flap 152 on the outer surface 148 with a connection plug 154 (shown and described in relation to FIGS. 12-14). The connection plug 154 may be inserted into the second socket port 150 to block the second socket port 150 when it is not in use. The fluid flow assembly may include a secondary clip 156 through which the insertion tube is inserted through. This secondary clip 156 may be used as known in the art and gives a point of reference in the various illustrations of the assembly 100.

The fluid flow assembly 100 includes a clip 200 for retaining the connection of the socket fitting 134 to the plug fitting 136 of the receptacle connector 134. The clip 200 includes a first connection end 202, a second connection end 204, and an elongated retaining portion 206. The first connection end 202 includes a first tube aperture 208 configured to be rotatably connected to one of the insertion tube 116 and a receptacle tube 122 (shown and described in relation to FIG. 3) through having the one of the insertion tube 116 and the receptacle tube 122 inserted through the first tube aperture 208. The second connection end 204 includes a tube connector 210 configured to be selectively and detachably connected around the other one of the insertion tube 116 and the receptacle tube 122. The elongated retaining portion 206 includes a first retaining end 222 and a second retaining end 224, the first retaining end 222 fixedly connected to and configured to bend in relation to the first connection end 202, and the second retaining end 224 fixedly connected to and configured to bend in relation to the second connection end 204 for retaining the connection between the socket fitting 134 and the plug fitting 136. FIG. 2 shows the clip 200 attached around the insertion tube 116 when the socket fitting 134 is not connected to the plug fitting 136.

The clip 200 may be formed of plastic, metal, or another flexible substance which will hold its' form, but provide some flexibility as would be known in the art. The clip 200 may be cut from a sheet of plastic, or another substance, and then bent into the desired form. The bending may be accomplished with machinery or tools known in the art and may be accomplished with or without heating the material. In other embodiments, the clip 200 may be molded, formed through a 3D printing process, or formed in any other process which would be known in the art.

Referring now to FIG. 3 an exemplary embodiment of the fluid flow assembly 100 is illustrated in a perspective view. The fluid flow assembly of FIG. 3 includes the socket fitting 134 and clip 200 of FIG. 2. Also illustrated is the plug fitting 136, the receptacle tube 122, and the fluid receptacle 110. The assembly 100 is illustrated to show another perspective view of the socket fitting 134 and clip 200 of FIG. 2. The fluid receptacle 110 may include a food bag 112 when the insertion is a gastronomy tube 106, or alternatively a urine collection bag 114 when the insertion is a catheter 108. Other embodiments include any fluid receptacle known in the art which would hold fluids to be transferred to, or which have been transferred from the living body 102.

The receptacle tube 122 may include a first receptacle tube end 124 fluidly connected to the plug fitting 136, and a second receptacle tube end 126 fluidly connected to the fluid receptacle 110. The receptacle tube 122 may be a long, hollow cylinder of plastic or another flexible material or any other connector known in the art for transferring fluids to and from the fluid receptacle 110.

The plug fitting 136 may include a plug 144 and a plug port 146. The plug 144 may be a tapered insertion device configured to be inserted into and be retained by the socket 138. The plug 144 may have a channel (not shown) running through it for flowing fluids from the receptacle tube 122 through the plug port 146, and to the socket fitting 134. The plug port 146 may include an aperture through which fluid flowing from or to the receptacle tube 122 into the pug fitting 136 may pass.

When it is desired to for fluid to flow to the living body 102 from the fluid receptacle 110, or from the fluid receptacle 110 to the living body 102, the insertion tube 116 may be inserted through the first tube aperture 208 of the clip 200. The plug fitting 136 may be connected to the socket fitting 134 through inserting the plug 144 into the socket 138. The clip may then be detachably connected to the receptacle tube 122 through the tube connection 210. In the embodiment where the insertion 104 is a gastronomy tube 106 and the fluid receptacle 110 is a nutrition bag 112, liquid nutrition may flow from the nutrition bag 112 into the receptacle tube 122 and to the plug port 146. The liquid nutrition may then flow through the plug port 146 and plug 144 to the socket fitting 134. The liquid nutrition may flow through the socket fitting 134 into the insertion tube 116, through the insertion tube 116 into the gastronomy tube 106 and into the stomach of the living body 102. In the embodiment where the insertion 104 is a catheter 108 and the fluid receptacle 110 is a urine collection bag 114, the direction of flow would reverse. The clip 200 may assist in ensuring the receptacle connector 128 stays connected. If the patient jerks suddenly or moves to far, the clip 200 may detach from the receptacle tube 122 allowing the receptacle connector 128 to disconnect and preventing the insertion 104 from being pulled from the patient's body.

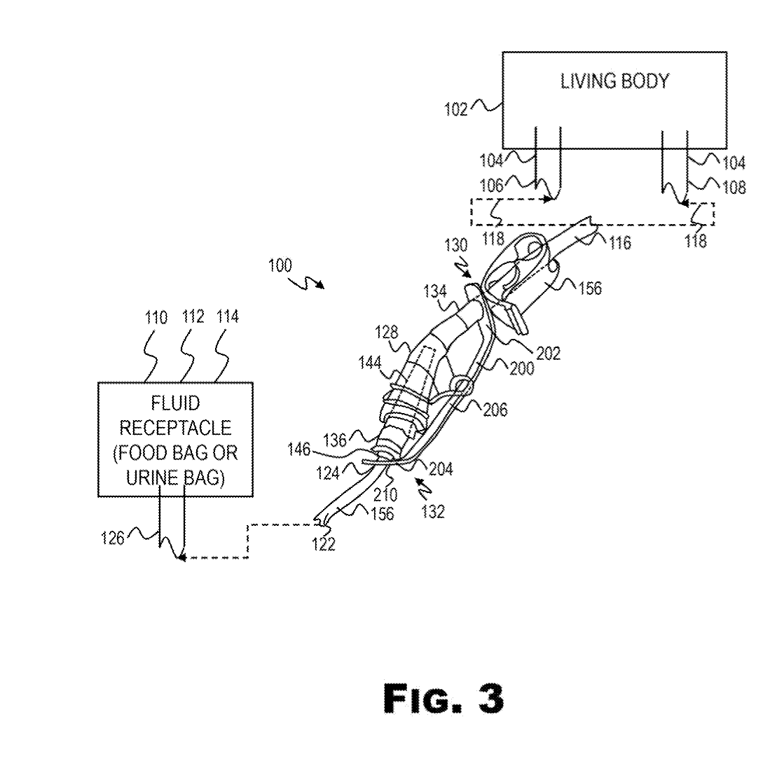

Referring now to FIG. 4, the fluid flow assembly 100 of FIG. 3 is illustrated from another perspective.

Referring now to FIG. 5, an alternative embodiment of the clip 200 is illustrated as part of the fluid flow assembly 100 in a perspective view. The embodiment of the clip 200 illustrated in FIGS. 2-4, the embodiment of the clip 200 illustrated here, and other embodiments of the clip 200 will be described in more detail in relation to FIGS. 7-10.

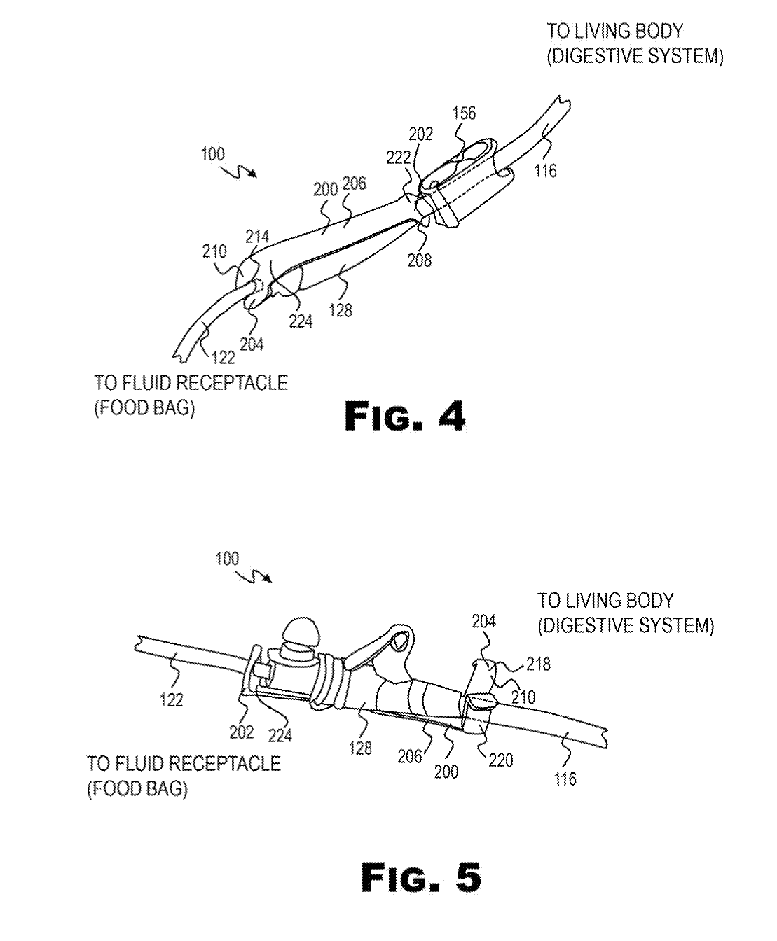

Referring now to FIG. 6, the fluid flow assembly of FIG. 5 is illustrated from another perspective.

Referring now to FIG. 7 an exemplary first embodiment of the clip 200 is illustrated in a perspective view. In this embodiment of the clip 200, the first connection end 202 is generally hexagonal in shape on the outside edges, and generally flat. The first connection end 202 may be bent forward (towards the viewer of the drawing) in relation to the elongated retaining portion 206. The first connection end 202 may include the first tube aperture 208 which may be generally circular in shape. The first tube aperture 208 may be sized such that the insertion tube 116 may be inserted through it, but the socket fitting 134 and the plug fitting 136 may not fit through it. This may ensure the first connection end 202 is retained on the body end 130 of the receptacle connector 128. Although not shown in this embodiment, the first connection end 202 may have a slit from the aperture to the outside edges to allow the first connection end 202 to be slid into connection with the insertion tube 116.

The second connection end 204, may be flat, generally circular, and bent forward from the elongated retaining portion 206. The second connection end 204 may include the tube connector 210. In the embodiment illustrated the tube connector 210 includes an outside edge 212, a second tube aperture 214, and a slit 216 connecting the outside edge 212 and the second tube aperture. The slit 216 may be slid around the receptacle tube 122 such that the receptacle tube 122 is inserted through the second tube aperture 214. Although the slit 216 is shown rather wide in this figure, depending on the flexibility of the material the clip 200 is formed from, the slit may be much narrower.

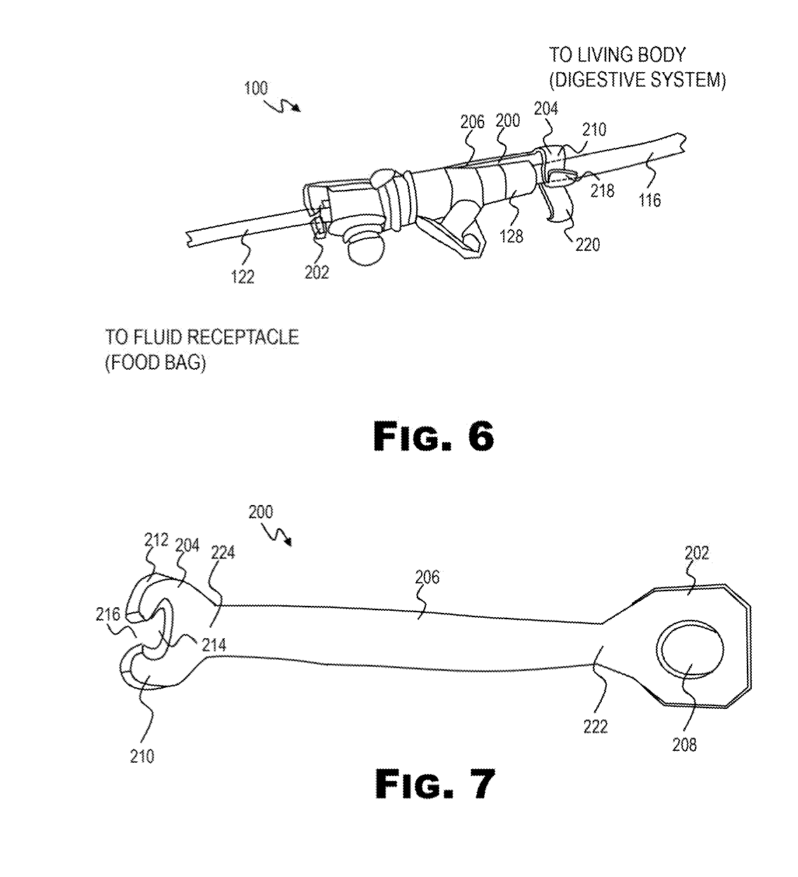

Referring now to FIG. 8, an exemplary second embodiment of the clip 200 is illustrated in a perspective view. In contrast to the embodiment of FIG. 7, the first connection end 202 is generally circular, as opposed to hexagonal. The first connection end 202 and the second connection end 204 may be different shapes in different embodiments.

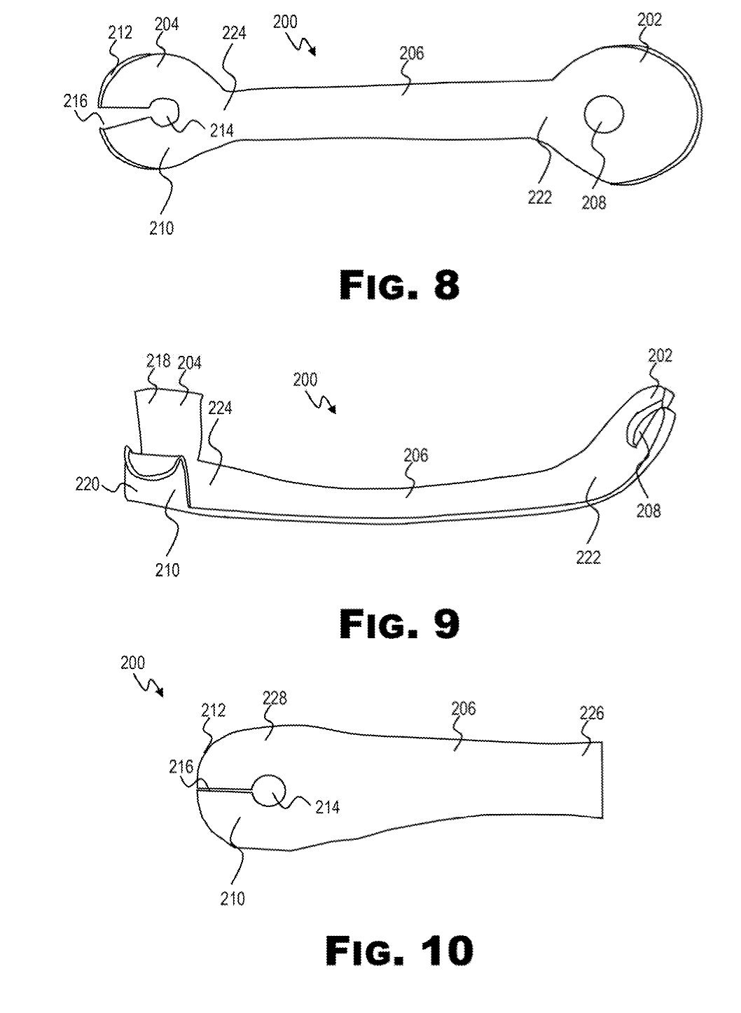

Referring now to FIG. 9, an exemplary third embodiment of the clip 200 is illustrated in a perspective view. In this embodiment the second connection end 204 and tube connector 210 include a first prong 218 and a second prong 220. The first prong 218 and the second prong 220 are bent together towards the elongated retaining portion 206. The receptacle tube 122 may be inserted between the prongs 218, 220. The prongs 218, 220 may have the flexibility and resilience to part to allow the receptacle tube 122 to be inserted, and to snap back in place to retain the receptacle tube 122. The first connection end 202 is illustrated as generally circular and including a slit.

Referring now to FIG. 10, an exemplary fourth embodiment of the clip 200 is illustrated in a perspective view. The clip 200 is configured to be fixedly attached to the outer surface 148 of the socket fitting 134, as will be illustrated in relation to FIGS. 12-14. In this embodiment, the elongated retaining portion 206 includes a socket connection end 226 instead of a first retaining end 222. The socket connection end 226 fixedly connects to the outer surface 148. The second connection end 204 and tube connector 210 are illustrated as generally circular with a generally circular second tube aperture 214, and a narrower slit 216 between the second tube aperture 214 and the outer edge 212. In other embodiments, the tube connector 210 may take different forms.

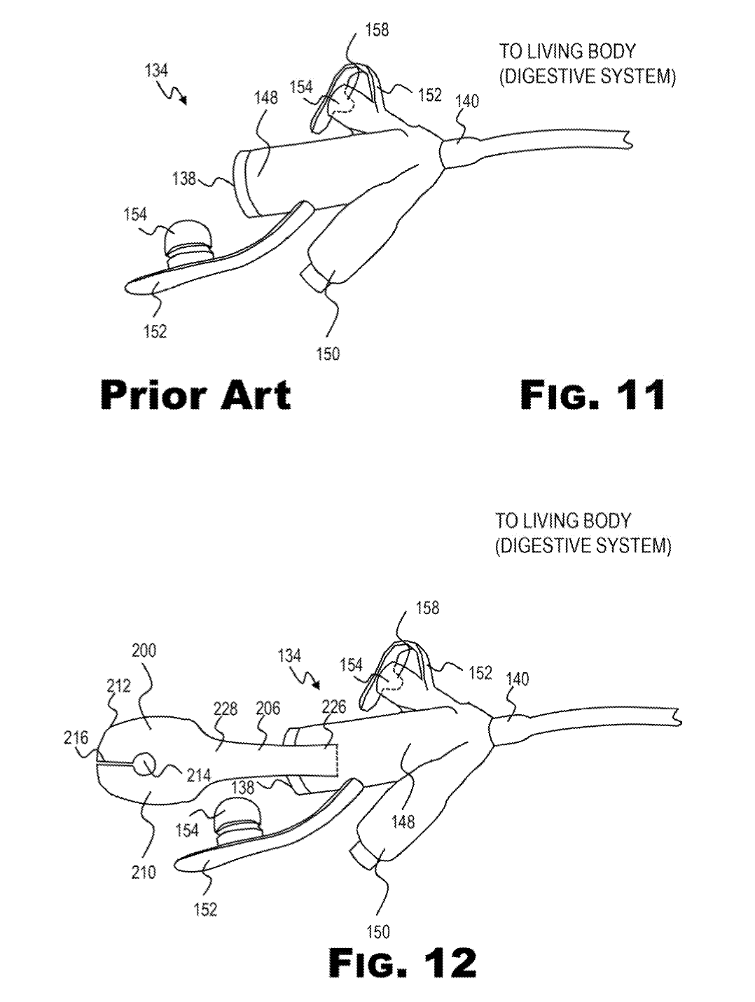

Referring now to FIG. 11, an exemplary socket fitting 134 from the prior art is illustrated in a perspective view. In this socket fitting 134 embodiment, a flap 152 is part of the outer surface 148 and includes an embodiment of the connection plug 154. A third socket port 158 with a flap 152 and connection plug 154 is also illustrated. The connection plugs 154 may selectively block the socket 138, and/or third connection port 158.

Referring now to FIG. 12, a first embodiment of an exemplary socket fitting 134 of the receptacle connector 128 with the clip 200 illustrated in FIG. 10 is illustrated in a perspective view. The socket connection end 226 is fixedly attached to the outer surface 148 approximately ninety degrees around the socket fitting 134 from the flap 152. The clip 200 may be fixedly attached to the outer surface with an adhesive after manufacture or during manufacture. The clip 200 may also be an integral part of the socket fitting 134 as manufactured. When the socket fitting 134 is connected to the plug fitting 136, the tube connector 210 may be detachably connected around the receptacle tube 122. In other embodiments, the tube connector may be detachably connected around the insertion tube 116 (where the plug fitting 136 is on the body side 130).

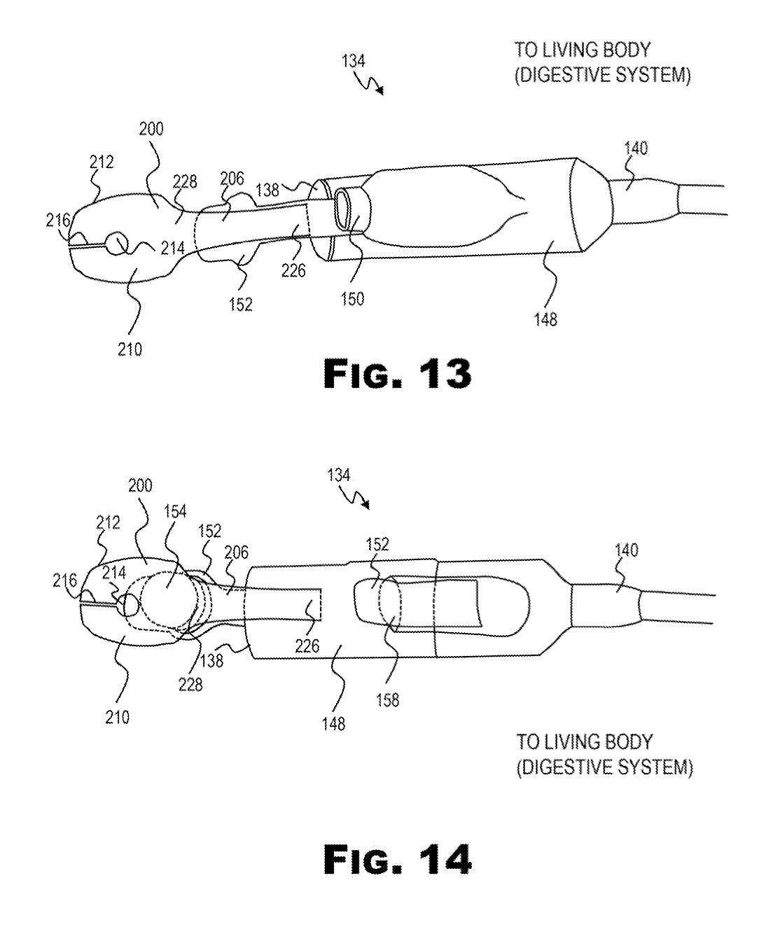

Referring now to FIG. 13, a second embodiment of an exemplary socket fitting 134 of the receptacle connector 128 with the clip 200 illustrated in FIG. 10 is illustrated in a perspective view. In this embodiment, the socket connection end 226 is fixedly attached to the flap 152.

Referring now to FIG. 14, a third embodiment of an exemplary socket fitting 134 of the receptacle connector 128 with the clip 200 illustrated in FIG. 10 is illustrated in a perspective view. In this embodiment, the socket connection end 226 is fixedly attached to the outer surface 148 approximately one hundred and eighty degrees around the socket fitting 134 from the flap 152

From the foregoing it will be appreciated that, although specific embodiments have been described herein for purposes of illustration, various modifications or variations may be made without deviating from the spirit or scope of inventive features described herein. Other embodiments will be apparent to those skilled in the art from consideration of the specification and figures and practice of the arrangements disclosed herein. It is intended that the specification and disclosed examples be considered as exemplary only.

I claim:

1. A clip for retaining the connection of a socket fitting to a plug fitting of a receptacle connector, one of the socket fitting and the plug fitting fluidly connected to an insertion in a living body through an insertion tube at a first connection port, and the other of the socket fitting and the plug fitting fluidly connected to a fluid receptacle through a receptacle tube at a second connection port, comprising:
a first connection end with a first tube aperture configured to be rotatably connected to one of the insertion tube and the receptacle tube through having the one of the insertion tube and the receptacle tube inserted through the first tube aperture;
a second connection end with a tube connector configured to be selectively and detachably connected around the other one of the insertion tube and the receptacle tube; and
an elongated retaining portion with a first retaining end and a second retaining end, the first retaining end fixedly connected to and configured to bend in relation to the first connection end, and the second retaining end fixedly connected to and configured to bend in relation to the second connection end.

2. The clip of claim 1, wherein the first connection end, the second connection end, and the elongated retaining portion are cut out of one sheet of a flexible material.

3. The clip of claim 1, wherein the first connection end, the second connection end, and the elongated retaining portion are molded as a single piece.

4. The clip of claim 1, wherein the first connection end, the second connection end, and the elongated retaining portion are formed as one piece with a three dimensional printer.

5. The clip of claim 1, wherein the second connection end includes an outside edge, a second tube aperture, and a slit running between the outside edge and the second tube aperture.

6. The clip of claim 1, wherein the second connection end includes a first prong and a second prong, both the first prong and the second prong bent inward towards the elongated retaining portion.

7. A socket fitting for a receptacle connector, the receptacle connector for fluidly connecting an insertion in a living body with a fluid receptacle and including a plug fitting, the plug fitting including a plug port and configured to be inserted into the socket fitting, the plug port configured to fluidly connect to the fluid receptacle through a receptacle tube, comprising:
a first socket port configured to fluidly connect to the insertion through an insertion tube;
an outer surface;
a clip with a socket connection end, a receptacle tube connection end, and an elongated retaining portion connecting the socket connection end to the receptacle tube connection end, the socket connection end fixedly attached to the outside surface, the tube connection end configured to be selectively and detachably connected to the receptacle tube.

8. The socket fitting of claim 7, wherein the outer surface includes a flap with a first socket port connection plug configured to be inserted into the first socket port, and wherein the clip is fixedly attached to the flap.

9. The socket fitting of claim 7, further including a second socket port and wherein the outer surface includes a flap with a port connection plug configured to be inserted into the second socket port, and wherein the clip is fixedly attached to the flap.

10. The socket fitting of claim 7, wherein the tube connection end includes an outside edge, a tube aperture, and a slit running between the outside edge and the tube aperture.

11. A fluid flow assembly for connecting an insertion within a living body with a fluid receptacle for collecting fluids through the insertion from the body or providing fluids through the insertion to the body, comprising:
an insertion tube with a first insertion tube end and a second insertion tube end, the first insertion tube end fluidly connected with the insertion;
a receptacle connector including;
a body end;
a receptacle end;
a socket fitting with a socket and a socket port, the socket port located on one of the body end and the receptacle end; and
a plug fitting including a plug port and a plug inserted into and fluidly connected with the socket, the plug port located at the other of the body end and the receptacle end;
a receptacle tube including a first receptacle tube end and a second receptacle tube end, the first receptacle tube end fluidly connected with one of the socket port and the plug port, the one of the socket port and the plug port located on the receptacle end, the second receptacle tube end fluidly connected with the fluid receptacle; and
a clip, including;
a first connection end with a tube aperture rotably connected around one of the insertion tube at the second insertion tube end and the receptacle tube at the first receptacle tube end;
a second connection end with a tube connector configured to be selectively and detachably connected around the other of the insertion tube at the second insertion tube end and the receptacle tube at the first receptacle tube end; and
an elongated retaining portion with a first retaining end and a second retaining end, the first retaining end fixedly connected to and configured to bend in relation to the first connection end, and the second retaining end fixedly connected to and configured to bend in relation to the second connection end; and
wherein the insertion tube second end is fluidly connected to the other of the socket port and the plug port, the other of the socket port and the plug port located on the body end.

12. The fluid flow assembly of claim 11, wherein the socket port is fluidly connected to the insertion tube second end, and the plug port is fluidly connected to the receptacle tube first end.

13. The fluid flow assembly of claim 11, wherein the plug port is fluidly connected to the insertion tube second end, and the socket port is fluidly connected to the receptacle tube first end.

14. The fluid flow assembly of claim 11, wherein the insertion is a gastronomy tube and the fluid receptacle is a nutrition bag for providing food through the gastronomy tube to the living body.

15. The fluid flow assembly of claim 11, wherein the insertion is a catheter and the fluid receptacle collects urine for disposal.

16. The fluid flow assembly of claim 11, wherein the second connection end includes an outside edge, a tube aperture, and a slit running between the outside edge and the tube aperture.

17. The fluid flow assembly of claim 11, wherein the second connection end includes a first prong and a second prong, both the first prong and the second prong bent inward towards the elongated retaining portion
